(19) United States
(12) Patent Application Publication
(10) Pub. No.: US 2004/0102406 A1
(43) Pub. Date: May 27, 2004

(54) Antisense oligonucleotides to inhibit angiogenesis and/or metastasis

(76) Inventors: Pnina Brodt, Montreal (CA); John Nip, Brooklyn, NY (US); Amy Wong, New York, NY (US)

Correspondence Address: KEOWN & ASSOCIATES, 500 WEST CUMMINGS PARK, SUITE 1200, WOBURN, MA, 01801, US

(21) Appl. No.: 10/312,752

(22) PCT Filed: Jun. 28, 2001
(86) PCT No.: PCT/CA01/00960

(51) Int. Cl. A61K 48/00; C07H 21/04

(52) U.S. Cl. 514/44; 536/23.2

(57) ABSTRACT

The present invention relates to a novel class of inhibitors of angiogenesis and/or metastasis that targets cell adhesion molecules, and more particularly to antisense phosphorothioate oligonucleotides directed to a subunit of an integrin vitronectin receptor to inhibit angiogenesis and/or metastasis. There is provided an antisense phosphorothioate oligonucleotide directed to one of a αᵥ, β₃ and β₅ subunit of an integrin vitronectin receptor. The antisense phosphorothioate oligonucleotide blocks synthesis of the integrin vitronectin receptor on a target cell, thereby inhibiting angiogenesis and/or metastasis. There is provided a method for blocking angiogenesis and/or metastasis in a patient, comprising delivering an efficient amount of such an antisense phosphorothioate oligonucleotide to the target cell of the patient, thereby blocking synthesis of the integrin vitronectin receptor on the target cell and blocking angiogenesis and/or metastasis.

### BACKGROUND OF THE INVENTION

(a) Field of the Invention

The present invention relates to a novel class of inhibitors of angiogenesis and/or metastasis that targets cell adhesion molecules, and more particularly to antisense oligonucleotides directed to a subunit of an integrin vitronectin receptor to inhibit angiogenesis and/or metastasis.

(b) Description of Prior Art

Angiogenesis, the growth of new blood vessels from pre-existing ones, is a process essential in normal physiological conditions including embryonic development, reproduction, placental development, inflammation, tissue remodeling and wound healing. Under these biological conditions, angiogenesis is a transient and highly regulated process. However, in pathological conditions, persistent deregulated angiogenesis occurs as a result of increased production of angiogenic stimulators and decreased production of negative regulators. This persistent and uncontrolled angiogenesis contributes to the pathogenesis of a variety of diseases such as diabetic retinopathy, rheumatoid arthritis and chronic inflammation, and plays a crucial role in the progressive growth and metastatic spread of tumors (reviewed in Folkman J., Shing Y. J Biol Chem 267:10931, 1992).

Angiogenesis is a multi-step process which involves an orderly sequence of events: (1) the basement membrane of the parent vessel is disrupted and endothelial cell processes penetrate through it into the perivascular tissue; (2) the endothelial cells migrate in the perivascular stroma towards the source of the angiogenic stimulus; (3) endothelial cells at the tip of the sprout continue to migrate and the endothelial cells at the mid-section of the growing capillary sprout undergo proliferation; (4) loop formation occurs as individual capillary sprouts anastomose with each other; (5) differentiation of the capillary sprouts is accompanied by capillary lumen formation; (6) blood flow slowly begins and synthesis of new basement membrane follows; (7) and finally, the formation of an entire capillary network (reviewed in Kumar R. et al. Int. J. Oncology 12:749-757, 1998).

In vitro, studies of angiogenesis usually employ cultures of large vessel and capillary endothelial cells. This required developing methods for isolation and maintenance of endothelial cells in long-term cultures. Experiments employing capillary endothelial cells demonstrated that all the information necessary to construct a capillary tube, to form branches, and to build an entire capillary network in vitro can be expressed by a single cell type, that is the vascular endothelial cells. To date, endothelial cells from various sources have been successfully isolated and cultured in vitro (Folkman J., Haudenschild C. C. Nature 288:551, 1980).

Human umbilical vein endothelial cells (HUVEC) have been used extensively as an in vitro model for angiogenesis. Although there are several advantages to employing these cells such as availability and relative Case of preparation (Maciag T. et al. J Cell Biol 91:420, 1981), many variables such as the different sources of umbilical cords and the age difference between the placentas can affect the properties of the isolated cells. In addition, the cells have relatively short life-spans in culture and long population doubling time of 92 h and may exhibit functional instability with increased passage Maciag T. et al. J Cell Biol 91:420, 1981).

Directional locomotion of endothelial cells is an early event in the formation of a capillary and dominates the process. It has been shown that an angiogenic signal can act as a chemotactic trigger and that angiogenic factors such as vascular endothelial growth factor (VEGF) can induce endothelial cells to secrete high concentrations of metalloproteinases (MMP-1, MMP-2, MMP-9) and plasminogen activators (uPA), which can degrade the basement membrane and facilitate cell migration through matrix barriers (Gross J. L. et al. Proc. Natl. Acad. Sci. USA 80:2623, 1983).

Endothelial cell migration in vitro can be studied by counting the number of cells that traverse a filter set in a Boyden chamber in response to a concentration gradient of an endothelial cell chemoattractant (Glaser B. M. et al. J. Cell. Biol. 60:673, 1980).

Another aspect of the angiogenic process is endothelial cell proliferation in response to angiogenic factors. Among all the angiogenesis-inducing factors identified to date, the best characterized include acidic fibroblast growth factor (aFGF), basic fibroblast growth factor (bFGF), platelet-derived endothelial cell growth factor (PD-ECGF), transforming growth factor-α (TGF-α) and vascular endothelial growth factor (Folkman J., Shing Y. J Biol Chem 267:10931, 1992) (reviewed in Kumar R. et al. Int. J. Oncology 12:749-757, 1998). bFGF is a potent stimulator of endothelial cell proliferation and motility. The role of bFGF in angiogenesis was confirmed using in vitro and in vivo models. Four bFGF-related high-affinity tyrosine kinase receptors were shown to be up regulated on activated endothelial cells, supporting the role of this factor in angiogenesis (Klagsbrun M., Baird A. Cell 67:229, 1991).

Under normal conditions, bFGF is not secreted in a soluble form but stored in the basement membrane and released only under restricted physiological conditions where neovascularization is required. Tumor cells can initiate bFGF-mediated angiogenesis by producing hydrolytic enzymes such as heparinase, collagenase and plasminogen activators which can mobilize bFGF from the extracellular matrix stores (Vlodavsky I. et al. Cancer Metastasis Rev 3:203, 1990).

VEGF is a member of a family of several related factors. The products of these related genes are glycosylated and dimerize after cleavage of their signal peptide. VEGF is highly conserved and has cross-species activity (Neufeld G. et al. Prog Growth Factor Res 5:89, 1994). Molecular cloning of the cDNA showed that VEGF shares an overall homology of 18% with platelet-derived endothelial cell growth factor (PD-ECGF). The human VEGF gene is organized into eight exons separated by seven introns. As a result of alternative splicing, four transcripts encoding monomeric VEGF consisting of 206 (V206), 189 (V189), 165 (V165) and 145 (V145) amino acid residues each have been identified. The most predominant isoform is V165, a diffusible secreted protein, which generates a 45 kDa peptide upon signal peptide cleavage (Tischer E. et al. J Biol Chem 266:11947, 1991).

VEGF expression is markedly increased in different tumors and both VEGF and its endothelial cell receptors (flt-1 and KDR) are frequently overexpressed in tumor-associated endothelial cells and in other pathological conditions characterized by angiogenesis such as wound healing, myocardial ischemia and inflammatory conditions.

Similarly to bFGF, VEGF can stimulate endothelial cell division but unlike other angiogenic growth factors, it is not mitogenic for non-endothelial cell types (e.g. epithelial cells, keratinocytes or fibroblasts) (Ferrara N. et al. Endocr Rev 13:18, 1992). It can also induce extracellular matrix degrading proteinases such as plasminogen activitors and the matrix metalloproteinase, interstitial (type I) collagenase. The induction of both plasminogen activators and collagenase by VEGF provides the necessary conditions for degradation of the extracellular matrix and subsequent migration of endothelial cells. VEGF can also upregulate expression of the integrin αᵥβ₃, which promotes endothelial cell migration on substrata consisting of αᵥβ₃ ligands such as vitronectin (Senger D. R. et al. Am J Path 149:293, 1996). Finally, VEGF has been shown to promote angiogenesis in many in vitro and in vivo assay systems (Ferrara N. et al. Endocr Rev 13:18, 1992). More direct evidence that VEGF plays a role in tumor angiogenesis has been derived from studies using specific monoclonal antibodies which inhibit VEGF-induced angiogenesis in vitro and in vivo (Kim K. J. et al. Nature 362:841, 1993).

Microvascular hyperpermeability also induced by VEGF may be essential for angiogenesis (Dvorak H. F. et al. Ann. NY Acad. Sci. 667:101, 1992, Nagy J. et al. Cancer Res 55:376, 1995) as it allows plasma proteins such as fibrinogen, fibronectin and albumin to extravasate from leaky blood vessels and form a new provisional matrix that promotes the inward migration of endothelial cells and fibroblasts (Dvorak H. F. et al. Ann. NY Acad. Sci. 667:101, 1992). Migrating endothelial cells and stromal fibroblasts act synergistically to form new blood vessels. The fibroblasts synthesize and secrete the matrix proteins, proteoglycans and glycosaminoglycans that make up the mature tumor stroma. Moreover, plasma fibrinogen that extravasates at tumor sites clots to form crosslinked fibrin which can provide a matrix substratum for cell adhesion and migration through its Arg-Gly-Asp (RGD) sequence. Other circulating RGD-containing plasma proteins that extravasate from leaky blood vessels at sites of tumor growth such as vitronectin and fibronectin, can also contribute to the generation of an extracellular matrix thereby promoting angiogenesis and the formation of new stroma (Yeo T. -K., Dvorak H. F. In Diagnostic Immunopathology pp 685-697. Raven Press, New York, 1995).

Angiogenesis is a critical component of tumorigenesis and metastasis. Following the establishment of a blood supply, a dormant tumor will begin to grow and has the potential to disseminate to distant sites via the haematogenous route. Angiogenesis facilitates tumor metastasis by providing an increased density of highly permeable blood vessels that have little basement membrane and fewer intercellular junctional complexes than normal blood vessels. They can therefore serve as a port of entry for the tumor cells into the systemic circulation where the cells can ultimately spread to distant organ sites. Because the number of metastases is likely to be proportional to the number of cells in the circulation, a decrease in angiogenesis at the primary site could lead to a reduction in the number of tumor cells which access the systemic circulation and as a result, decrease the number of metastatic colonies in distant target organs. The process of metastasis is therefore considered to be angiogenesis-dependent (Folkman J., Shing Y. J Biol Chem 267:10931, 1992).

The process of angiogenesis, is dependent on endothelial cell attachment, spreading and motility on extracellular matrix (ECM) proteins.

Adhesion to ECM is mediated by integrin transmembrane heterodimers which consist of non-covalently associated α and β subunits. To date fourteen a subunits and eight β subunits have been identified (e.g. αᵥ, α₆, α₄). Among them, the αᵥ subunit is known to associate with three different β subunit namely, β₁, β₃ and β₅ (Cheresh D. A. Adv. Mol. Cell. Biol. 6:225, 1993).

Both αᵥβ₃ and αᵥβ₅ have been implicated in angiogenesis (Brooks P. C. et al. Science 264:569, 1994a, Friedlander M. et al. Science 270:1500, 1995). Integrin αᵥβ₃ recognizes the Arg-Gly-Asp (RGD) sequence found within a number of extracellular matrix proteins including fibronectin, vitronectin, type I collagen, denatured type IV collagen (a.k.a. gelatin), Von Willibrand's factor, osteopontin, and adenovirus penton base (Cheresh D. A. Adv. Mol. Cell. Biol. 6:225, 1993). Although the expression of the integrin αᵥβ₃ in normal quiescent endothelial cells is low, it is upregulated on proliferating endothelial cells in the process of angiogenesis. Upregulation of the αᵥβ₃ integrin has been observed in newly forming blood vessels in a variety of tissues (Brooks P. C. et al. Science 264:569, 1994a), and pathological conditions (Friedlander M. et al. Proc. Natl. Acad. Sci. USA 93:9764, 1996).

Angiogenesis depends on the stimulation of quiescent endothelial cells by angiogenic factors to express the integrin αᵥβ₃ and also on the interactions of αᵥβ₃ with its ligands (Brooks P. C. et al. Science 264:569, 1994a). In addition, it was demonstrated that the type IV collagenase (MMP-2) binds directly to integrin αᵥβ₃ and is localized on the surface of invasive tumor cells or activated endothelial cells. This localization provides migratory cells with a mechanism for coordinated matrix degradation and cellular motility, thereby facilitating cellular invasion (Brooks P. C. et al. Cell 85:683, 1996). In addition, αᵥβ₃ is a survival factor for endothelial cells (Brooks P. C. et al. Cell 749:1157, 1994b). The highly restricted expression of αᵥβ₃, the upregulation of its expression during angiogenesis and its ability to mediate endothelial cell survival and migration make it a desirable target for anti-angiogenic therapy. Indeed monoclonal antibodies and synthetic peptides which block αᵥβ₃ function were shown to suppress ingrowth of blood vessels to tumor implants and to induce tumor regression in vivo (Brooks P. C. et al. Cell 749:1157, 1994b). There are several advantages to endothelial cell-directed therapy.

a) endothelial cells are easily accessible from the circulation.

b) in contrast to tumor cells which are genetically unstable and which may eventually develop drug resistance, endothelial cells are genetically stable and have a low mutational rate. In fact, the ability to circumvent the problems associated with acquired drug resistance in tumors has been demonstrated with the use of anti-angiogenic agents such as endostatin (Boehm T. et al. Nature 390:404, 1997).

Recently, new strategies based on genetic manipulation of endothelial cells as means of inhibiting angiogenesis have been developed. They are based on the transfer of DNA to target cells to produce therapeutic effects. One such group of anti-angiogenesis inhibitors that have shown great promise are the antisense oligodeoxynucleotides (ODN).

Antisense oligodeoxynucleotides are a novel class of therapeutic drugs which are designed to block protein expression and function in a highly specific manner.

Inhibition of gene expression by antisense ODN relies on their ability to hybridize to a complementary messenger RNA (mRNA) sequence through Watson-Crick base pairing and consequently prevent translation of the mRNA to its protein product (Milligan J. F. et al. J Med Chem 36:1923, 1993). Several mechanisms of antisense ODN action have been described. They include the activation of RNase H, which cleaves the RNA strand of the RNA-DNA duplex (Giles R. V. et al. Antisense Res. Dev. 5:23, 1995), translation arrest triggered as antisense ODN hybridize to the target mRNA molecule and block ribosome movement and mRNA translation and prevention of ribosome assembly on the mRNA strand. Inhibition of RNA processing by the prevention of intron splicing, disruption of secondary or tertiary RNA structure and inhibition of 5′ capping or 3′ polyadenylation, are other proposed mechanisms of antisense ODN actions. Ultimately, the mechanism of action may depend on the specific ODN chemistry and the specific mRNA sequence to which the oligodeoxynucleotide sequence hybridizes (reviewed in Wagner R W Nature, 372:333-335, 1994).

Several strategies have been employed to increase the resistance of ODN to exo- and endonucleases and thereby extend their half-life. They are based mainly on chemical modifications to the phosphate backbone, including substitution of one of the non-bridging oxygen atoms on the phosphate group with a methyl group or a sulfur atom, to produce methyl-phosphonate and phosphorothioates, respectively (Manoharan M. et al. Annals NY Acad Sci 660:306, 1992). In addition to increasing ODN resistance to degradation by nucleases, phosphorothioate oligonucleotides also support RNase H-mediated hydrolysis of the target mRNA (Monia B. P. et al. J Biol Chem 268:14514, 1993). The stability of phosphorothioate oligonucleotides varies depending on the cell line investigated. In cell-based assays, phosphorothioate oligonucleotides have been reported to reduce expression of the target mRNA for 24-48 hours (Monia B. P. et al. J Biol Chem 271:14533, 1996a) and longer lasting suppression could be achieved by repeated administration of oligonucleotides (Altman K. -H. et al. Chimia 50: 168, 1996).

Among the successful applications of antisense ODN are the use of c-myb phosphorothioate ODN to inhibit human leukaemia-cell growth (Hijiya N. et al. Proc. Natl. Acad. Sci. USA 91:4499, 1994) bcl-2 phosphorothioate ODN to suppress growth of B-cell lymphoma cells in SCID mice (Cotter F. E. et al. Oncogene 9:3049, 1994) and the intravenous administration of a c-raf kinase phosphorothioate ODN to reduce growth of several human tumors implanted in nude mice (Monia B. P. et al. Nat Med 2:668, 1996b). In addition, antisense oligonucleotides have been utilized to downregulate expression of endothelial cell adhesion molecules as means of treating cancers. For instance, an intraperitoneal administration of an antisense ODN for intercellular adhesion molecule 1 (ICAM-1) was shown to reduce melanoma metastasis in mice (Miele M. E. et al. Exp Cell Res 214:231, 1994).

Based on cell-culture and animal studies, several antisense oligonucleotides have recently been approved for clinical trials, they include GEM 91-a 25-mer antisense phosphorothioate ODN which binds to the gag region of HIV RNA (Lisziewicz J. et al. Proc. Natl. Acad. Sci. USA 91:7942, 1994), Formivirsen, a phosphorothiate ODN currently in Phase III trials as a locally administered treatment for CMV retinitis (Kisner D. Fourth Annual International Symposium on Oligonucleotide and Gene Therapy-Based Antisense Therapeutics with New Application for Genomics, San Diego, 1997) and, ISIS 2302, an ICAM-1 antisense ODN, which is currently in Phase II trials for the treatment of inflammatory conditions such as Crohn's disease, ulcerative colitis, and rheumatoid arthritis (Yacyshyn B. R. et al. Gastroenterology 114:1133, 1998).

During the course of angiogenesis, endothelial cells invade the basement membrane and migrate to the perivascular tissue stroma. The degradation of basement membrane macromolecules is achieved through the elaboration of several proteolytic enzymes. One proteinase critical to the process is the urokinase plasminogen activator (uPA), a serine proteinase which is activated when bound to its membrane-linked receptor (uPAR). uPA converts serum plasminogen to plasmin which in turn, can degrade a broad spectrum of extracellular matrix proteins including fibronectin and laminin. Plasmin can also activate several collagenases, which can degrade types I, II, III and IV collagens. In endothelial cells, production of uPA and UPAR were shown to rise in response to angiogenic factors (reviewed in Reuning U. et al. Int. J. Oncology 13:893-906, 1998).

It would therefore be highly desirable to develop inhibitors of angiogenesis that would target endothelial cell adhesion molecules and ECM degrading proteolytic enzymes.

### SUMMAY OF THE INVENTION

One aim of the present invention is to provide a novel class of angiogenesis inhibitors that targets cell adhesion molecules.

In accordance with the present invention there is provided an antisense oligonucleotide directed to αᵥ subunit of an αᵥβ₃ or αᵥβ₅ integrin vitronectin receptor. The antisense oligonucleotides block synthesis of the integrin vitronectin receptor on a target cell, thereby inhibiting angiogenesis and/or metastasis.

Preferably, the integrin vitronectin receptor it targets is αᵥβ₃.

Preferably, the antisense oligonucleotide has a length of from 17 to 25 bases. More preferably, the antisense oligonucleotide has a length of 18 bases and is complementary to bases 31-48 of a sequence of a human αᵥ subunit encoding a signal peptide of the αᵥ molecule.

The antisense oligonucleotide preferably has a sequence as set forth in SEQ ID NO:2.

The antisense oligonucleotide is preferably an antisense phosphorothioate oligonucleotide.

In accordance with another aspect of the present invention, there is provided a method for blocking delivering an efficient amount of such an antisense oligonucleotide to the target cell of the patient, thereby blocking synthesis of the integrin vitronectin receptor on the target cell and blocking angiogenesis.

The target cell may be a vascular endothelial cell.

The patient may have a disease selected from the group consisting of diabetic retinopathy, rheumatoid arthritis, chronic inflammation and cancer.

In accordance with yet another aspect of the present invention, there is provided a method for blocking metastasis from a primary site of a tumor in a patient. The method comprises delivering an efficient amount of such an antisense oligonucleotide to the target cell of the patient, thereby inhibiting expression of adhesion molecules by the target cell and blocking metastasis from the primary site of the tumor in the patient.

Still in accordance with the present invention, there is provided the use of the antisense of the present invention for blocking angiogenesis and for the manufacture of a medicament for blocking angiogenesis.

The effect of αᵥ antisense ODN on expression of αᵥ in human endothelial cells and its effect on endothelial cell migration were investigated. It was rationalized that if αᵥ antisense oligonucleotides suppress αᵥ synthesis in endothelial cells and reduce endothelial cell migration and proliferation, they can serve as inhibitors of angiogenesis, since these three parameters are critical to the process of angiogenesis. Also, based on previous studies it was rationalized that the suppression of αᵥβ₃ expression will reduce synthesis of uPAR thereby reducing plasmin production and degradation of extracellular matrix, an essential step in angiogenesis. Successful “cell adhesion-targeted therapy” represents a major new approach for the prevention of human diseases involving abnormal angiogenesis such as tumor growth and metastasis.

### BRIEF DESCRIPTION OF THE DRAWINGS

FIG. 1 illustrates the identification of an αᵥ antisense oligonucleotide with a potent inhibitory effect on αᵥ gene expression;

FIG. 2 illustrates the dose-dependent inhibition of αᵥ expression by AS2;

FIG. 3 illustrates the specificity and variability in the response to αᵥ antisense ODN between different endothelial cell cultures;

FIG. 4 illustrates the dose-dependent inhibition of cell migration by αᵥ antisense oligonucleotides;

FIG. 5 illustrates the inhibition of HUVEC migration by αᵥ antisense ODN; and

FIGS. 6A and 6B illustrate the inhibition of bFGF-induced DNA synthesis in HUVEC by αᵥ antisense ODN.

### DETAILED DESCRIPTION OF THE INVENTION

The effect of a, antisense phosphorothioate oligodeoxynucleotides (ODN) on αᵥβ₃ expression and on endothelial migration was assessed using human umbilical vein endothelial cells (HUVEC). It was found that αᵥ antisense phosphorothioate ODN reduced αᵥβ₃ expression in endothelial cell cultures and this resulted in a dose-dependent decrease in endothelial cell migration. The αᵥ antisense phosphorothioate ODN therefore represents a novel class of angiogenesis inhibitors.

Primary cultures and early passages of human umbilical vein endothelial cells (HUVEC) were used to establish an in vitro angiogenesis assay and to assess the effect of αᵥ antisense ODN on two parameters of the angiogenic process, namely cellular migration and proliferation. The results show that HUVEC cells respond to motogenic and mitogenic signals triggered by VEGF and bFGF, respectively and that pre-treatment of the cells with αᵥ antisense ODN inhibited these responses.

The integrins expressed by HUVEC include α₂β₁, α₃β₁, α₅β₁ and αᵥβ₃ (Luscinskas F. W., Lawler J. FASEB J 8:919, 1994). αᵥβ₃ was identified as a marker of angiogenesis on vascular cells in various in vivo and in vitro models (Brooks P. C. et al. Science 264:569, 1994a). αᵥβ₃ can serve as an effective target for anti-angiogenic therapy, based on the present investigation into the potential anti-angiogenic effect of αᵥ antisense ODN.

Three eighteen-base antisense phosphorothioate ODN, AS1, AS2 and AS3 as shown in Table 1, selected to have no homology with DNA sequences of other known integrin subunits, α₅ in particular, and previously shown to have αᵥ suppressing effects in a melanoma model, were first used. At a concentration of 40 μM, AS2 reduced αᵥ expression by 78% but AS1 and AS3 had no effect. This inhibitory effect was confirmed with several HUVEC cultures and was dose-dependent. A sense ODN control had no effect, suggesting the reduction in αᵥ expresssion was due to specific effects of AS2. The reason for the lack of effect of AS1 and AS3 on αᵥ synthesis may be that not all sites on the target mRNA are equally accessible to ODN. For example, when the effects of a series of synthetic oligonucleotides complementary to the 5′ non-coding and coding regions of rabbit beta-globin mRNA on endogenous protein synthesis, was tested using a rabbit reticulocyte cell-free translation system, it was found that the sites most sensitive to inhibition are at the start of the 5′ noncoding region and a sequence including the initiation codon and several upstream bases. Similarly, when antisense pentadecamers complementary to different sequences between the cap and AUG initiation codon of the c-myc mRNA were tested, it was found that the activity of antisense sequences complementary to cap-region sequences was 2-3 fold higher than the activity of an initiation codon antisense sequence. These observations suggest that the efficacy of an antisense ODN sequence depends on its site of hybridization on the mRNA sequence.

AS2 is complementary to the sequence spanning bases 31-48 of the human αᵥ subunit, which encodes the signal peptide of the αᵥ molecule (Suzuki S. et al. J Bio Chem 262:14080, 1987, Fitzgerald L. et al. Biochem 26:8158, 1987). It is possible that the corresponding αᵥ mRNA sequence has a higher binding efficiency for AS2 than those targeted by AS1 and AS3, although this was not the case in human melanoma cells.

Having determined the optimal conditions for HUVEC migration, the effect of AS2 on migration was analyzed. It was found that the inhibitory effect on HUVEC migration exhibited by AS2 was dose-dependent and the reduction reached up to 95% at a concentration of 40 μM. An ODN sequence complemetary to mouse αᵥ also decreased the cell migration, but the reduction was lower in magnitude than the reduction caused by AS2.

DNA synthesis in HUVEC (an indirect measure of all division) was reduced by up to 78% with 40 μM AS2 while mouse αᵥ antisense reduced it up to 62%.

It was observed that individual HUVEC cultures varied in respect to reductions in αᵥ levels resulting from αᵥ antisense ODN treatment. This variability may be due to difference in ODN uptake by different cell cultures. As ODN are negatively charged, their diffusion across the membrane lipid bilayer is highly inefficient and may be as low as 1-2% of the total oligonucleotides. The efficiency of uptake can however be increased by addition of cationic liposomes (Bennett C. F. et al. J. Lip. Res. 3:85, 1993).

It was shown that the length of time that vascular endothelial cells are maintained in culture may affect some of their properties including prostacyclin release, angiotensin-converting enzyme activity, gene expression and cell cycle kinetics (Goldsmith J. C., et al. Lab Invest 51:643, 1984). To standardize assay conditions and increase reproducibility, HUVEC in the present study were used only between first and fifth in vitro passages, at which time they were discarded. This necessitated the use of fresh endothelial cell cultures prepared from newly obtained umbilical cords throughout this study. The inconsistency observed in the effects of αᵥ antisense ODN on αᵥβ₃ expression and function may also have been related to variability between the cords obtained. Diverse factors such as the age of the fetus, mother's age, mother's health and clinical history, and environmental factors may have contributed to differences in αᵥβ₃ expression, various cell properties including cell permeability, cell doubling time, expression of other relevant adhesion molecules, and growth factor and growth factor receptor levels expressed by the cells. These factors among others may have contributed to the variability both in cell surface αᵥ expression following antisense ODN treatment and in the functional impact of this treatment. Future In vitro angiogenesis studies may be facilitated by the use of human endothelial cell lines.

The factors responsible for the non-specific effects of murine αᵥ antisense ODN are not presently clear. The results indicate however that a murine ODN sequence is not an optimal control for the study of human cells.

Taken together, the present results show that αᵥ antisense ODN can significantly reduce cell surface αᵥ levels on endothelial cells and inhibit cellular functions essential for angiogenesis. Based on these results, the ODN disclosed herein as embodiments of the present invention and others of the present invention have potential clinical applications.

Moreover, the present results coupled with the fact that antagonists of αᵥβ₃ can block angiogenesis show that targeting of integrins provides an effective anti-angiogenic approach. Because a large variety of adhesion molecules are expressed with great specificity on different tumor cells and at different stages of the angiogenic process (e.g. αᵥβ₃ is only expressed upon angiogenic stimulation), it is possible to selectively interfere with these adhesive processes without blocking normal established adhesive interactions in the same tissue. Such “anti-adhesive therapies” may represent a major new approach to the treatment of malignant tumors.

The present invention will be more readily understood by referring to the following examples which are given to illustrate the invention rather than to limit its scope.

In the following examples, umbilical cords for obtaining the human umbilical vein endothelial cell (HUVEC) cultures were obtained after normal delivery from the Surgical Pathology Laboratory of the Royal Victoria Hospital (Montreal, Quebec, Canada). They were stored at 4° C. in a saline solution containing 238 mg/ml N-2-hydroxyethylpiperazine-N′-2-ethanesulfonic acid (HEPES) (Gibco, Burlington, Ontario, Canada), 2 mg/ml anhydrous D-glucose (Sigma, Oakville, Ontario, Canada), and 0.3 mg/ml KCl (Fisher, Nepean, Ontario, Canada). Umbilical cord veins were cannulated and flushed with Ringer's solution to remove all traces of blood and thrombus, and then the luminal surfaces of the veins were filled with 0.1% collagenase (Sigma) in phosphate-buffered saline (PBS) (Gibco) and incubated for 15 min at 37° C. to detach endothelial cells. The cells were collected by flushing the veins with Medium-199 (Gibco) and centrifuged for 7 min at 1,000 rpm. Pellets were resuspended in Medium-199 (basic medium) supplemented with 20% fetal calf serum (FCS) (Wisent, St-Bruno, Quebec, Canada), 238 mg/ml HEPES (Gibco), 10,000 U/ml penicillin, 10,000 μg/ml streptomycin, 29.2 mg/ml L-glutamine (all purchased from Gibco), and with 1.5 mg/ml endothelial cell growth supplement (ECGS), and 1,500 U/ml heparin (both obtained from Sigma) (HUVEC medium). Cells isolated in this manner were previously identified as >90% endothelial in origin as confirmed by morphology (cobblestone-like) and positive immunofluorescence staining with an antibody to von Willebrand factor (McGill S. N. et al. World J Surg 22:171, 1998). Isolated endothelial cells were plated onto 75-cm² tissue culture flasks (Sarstedt, St-Leonard, Quebec, Canada) which were pre-coated with 0.3% gelatin (Fisher) to improve cell attachment, and incubated at 37° C. in a 5% CO₂ incubator. The next day, cells were gently washed twice with Medium-199 and fresh HUVEC medium was added. New culture medium was replenished on alternate days. HUVEC cultures of 80-90% confluency were used between the first and fifth passages.

The effects of the following three αᵥ antisense phosphorothioate oligonucleotide sequences on αᵥ expression by HUVEC were analyzed.

TABLE 1
αᵥ antisense phosphorothioate oligonucleotide sequences

| | | | |
|---|---|---|---|
| Oligonucleotide | Sequence | Complementarity | SEQ ID NO |

| | | | |
|---|---|---|---|
| AS1 | 5′-TCAGCATCAATATCTTGT-3′ | To bases 563- | SEQ ID NO:1 |
| | | 580 of the human | |
| | | αᵥ subunit | |
| | | sequence | |
| AS2 | 5′-AAGCCATCGCCGAAGTGC-3′ | to bases 31-48 of | SEQ ID NO:2 |
| | | the human αᵥ | |
| | | subunit sequence | |
| AS3 | 5′-GACTGTCCACGTCTAGGT-3′ | to bases 136-153 | SEQ ID NO:3 |
| | | of the human αᵥ | |
| | | subunit sequence | |
| Control | 5′-GCACTTCGGCGATGGCTT-3′ | Sense of AS2 | SEQ ID NO:4 |
| ISIS 16205 | 5′-CAAGGTCGCACACCACCTGC-3′ | to the mouse αᵥ | SEQ ID NO:5 |
| | | sequence with no | |
| | | homology to the | |
| | | human αᵥ | |
| | | sequence | |

Initially, these oligonucleotides were used to determine their effect on αᵥ and uPAR expression in human melanoma cells. The oligonucleotides were synthesized by the Sheldon Biotechnology Center (Montreal, Quebec, Canada) and purified three times using ethanol precipitation (2.5 vol of ethanol and 1/4 vol of 10 M ammonium acetate). Subsequently, the following oligonucleotides were obtained from ISIS Pharmaceuticals (Carlsbad, Calif., USA): ISIS 15630 (AS 2) described above and, as a control, ISIS 16205, as mentioned in Table 1.

Rat MAb 69-6-5 to human integrin subunit αᵥ (Lehmann M. et al. Cancer Res 54:2102, 1994, a gift from J. Marvaldi, Laboratoire de Biochimie Cellulaire, Universite d'Aix-Marseille, Marseille, France) was used as a primary antibody. A peroxidase-conjugated goat anti-rat immunoglobulin was used as a secondary antibody (Jackson ImmunoResearch Laboratories Inc., West Grove, Pa., USA).

Student's t test was used to analyze migration and thymidine incorporation data.

### EXAMPLE I

### Identification of an αᵥ antisense oligonucleotide with a potent inhibitory effect on cell surface αᵥ gene expression

Three antisense oligonucleotides sequences shown to suppress αᵥ expression in human melanoma cells are herein tested for their effect on αᵥ expression in an early passage HUVEC culture.

Cell Treatment with Oligonucleotides

HUVEC monolayers of 80-90% confluency were dispersed with 0.05% trypsin-EDTA (Sigma) and plated in 0,3% gelatin-coated 25-cm² tissue culture flasks (Sarstedt). The cells were allowed to spread in HUVEC medium. After 4˜5 h, oligonucleotides were added at the desired concentrations and this was repeated 24 h later for a total incubation time of 2 days at which time the oligonucleotides were removed and fresh HUVEC medium was added.

Quantitation of Cell Surface αᵥ Expression

Expression of cell surface αᵥ was assessed by the Enzyme-linked immunosorbent assay (ELISA). Cells were treated with oligonucleotides for 48 h in 0.3% gelatin-coated 96-well tissue culture plates (Sarstedt) in HUVEC medium. The cells were washed five times with PBS and non-specific protein binding sites were blocked with 1% bovine serum albumin (BSA) (Boehringer Mannheim, Laval, Quebec, Canada) in PBS for 1 h at room temperature. The cells were fixed with 0.125% glutaraldehyde (Fisher) in PBS for 2 min and rinsed five times with PBS containing 0.1% BSA (assay buffer). To each well, 10 μg/ml MAb 69-6-5 diluted in assay buffer were added for a 90-min incubation at room temperature. Unbound antibody was removed by washing the cells five times with assay buffer. A peroxidase-conjugated goat anti-mouse IgG diluted 1:1000 in assay buffer was used as a secondary antibody and incubated with the cells for 60 min at room temperature. Unbound antibody was removed by 5 washings with assay buffer. A calorimetric reaction was initiated with ABTS (2,2′-azino-bis (3-ethylbenzthiazoline-6-sulfonic acid)) (Boehringer Mannheim) as a substrate and stopped 8-10 min later by the addition of 0.05% sodium azide (Fisher). Color intensity was measured with a ThermoMAX™ Microplate reader (Molecular Devices Corporation, Menlo Park, Calif., USA) at a wavelength of 405 nm. The program used to analyze the data was SOFTmax™ 2.32 Software package for the MAXline™ Microplate Readers (Molecular Devices Corporation).

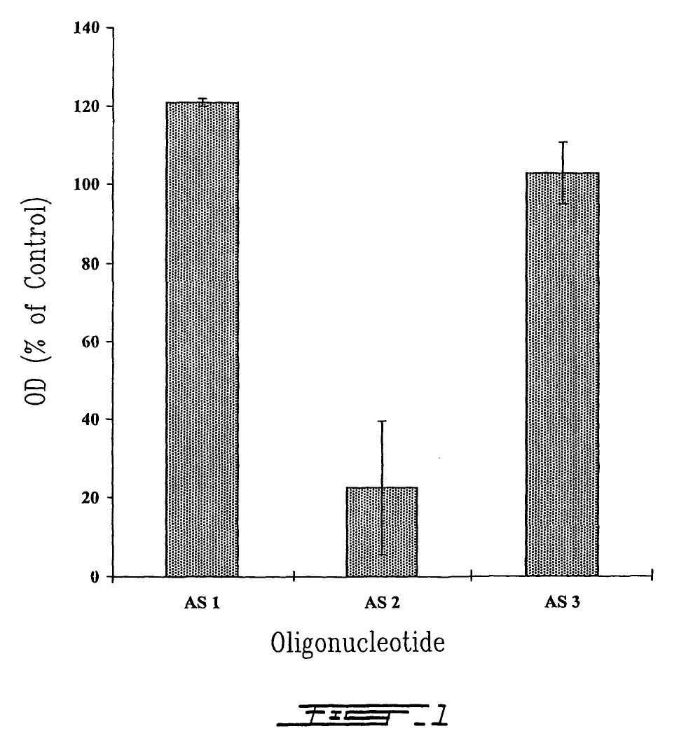

Results shown in FIG. 1 demonstrate that AS2 had the most potent inhibitory effect on cell surface αᵥ expression. At a concentration of 40 μM, the level of αᵥ decreased by 78%. Neither of the other two ODN, i.e. AS1 and AS3, reduced αᵥ expression. AS2 was therefore selected for all subsequent studies. In FIG. 1, HUVEC were treated with αᵥ antisense phosphorothioate oligonucleotides AS1, AS2 and AS3 at a concentration of 40 μM for 48 h at 37° C.; αᵥβ₃ expression was measured by ELISA; cells were washed with PBS and fixed with 0.125% glutaraldehyde; to each well, 50 μl of 0.1% BSA (negative control) or MAb 69-6-5 (10 μg/ml) were added for a 90 min incubation followed by a 60 min incubation with a peroxidase-conjugated goat anti-mouse antibody (1:1000); ABTS was used as a substrate and the color intensity was measured at 405 nm; results are expressed as percent expression relative to untreated cells and based on experiments done in triplicates.

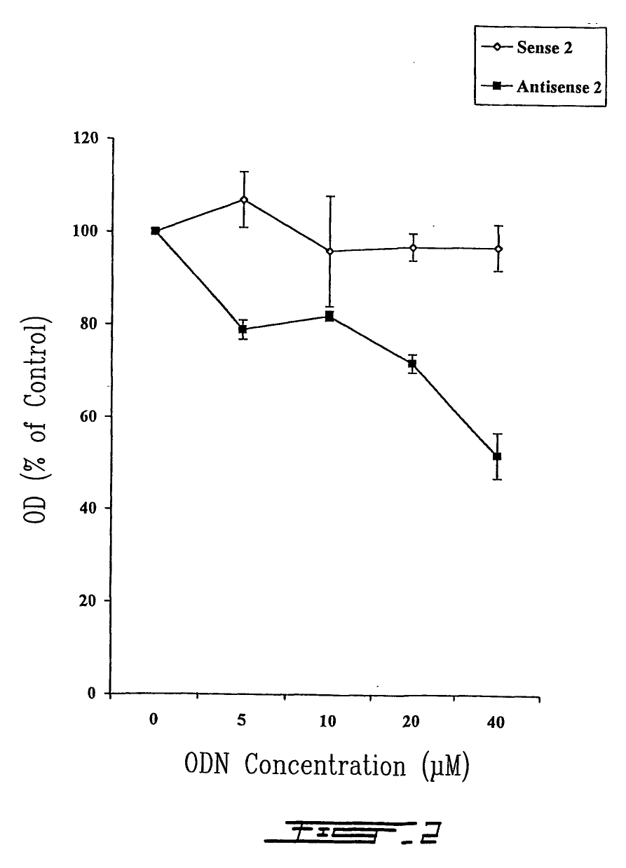

The inhibitory effect of AS2 on αᵥ expression was confirmed with a second HUVEC culture, as shown in FIG. 2. With these cells, the inhibition was shown to be dose-responsive and reached a maximum of 50% at a concentration of 40 μM ODN. A sense ODN control showed no effect. In FIG. 2, HUVEC were treated with AS2 at concentrations of 5-40 μM for 48 h at 37° C.; αᵥβ₃ expression was measured by ELISA as described with respect to FIG. 1; results are expressed as percent expression relative to untreated cells; and a phosphorothioate 18-base sense sequence corresponding to AS2 (Sense 2) was used as the control ODN.

In addition, antisense-treated cells lost adhesiveness and became rounded in morphology probably as a consequence of the reduction in αᵥ expression. This loss of adhesiveness of antisense ODN-pretreated endothelial cells was further confirmed by the yield of cells harvested after ODN treatment relative to untreated cells.

### EXAMPLE II

### Variability in Response to αᵥ Antisense ODN Between Different Endothelial Cell Cultures

The effect of AS2 on αᵥ expression is herein examined in multiple cultures derived from individual cords, simultaneously.

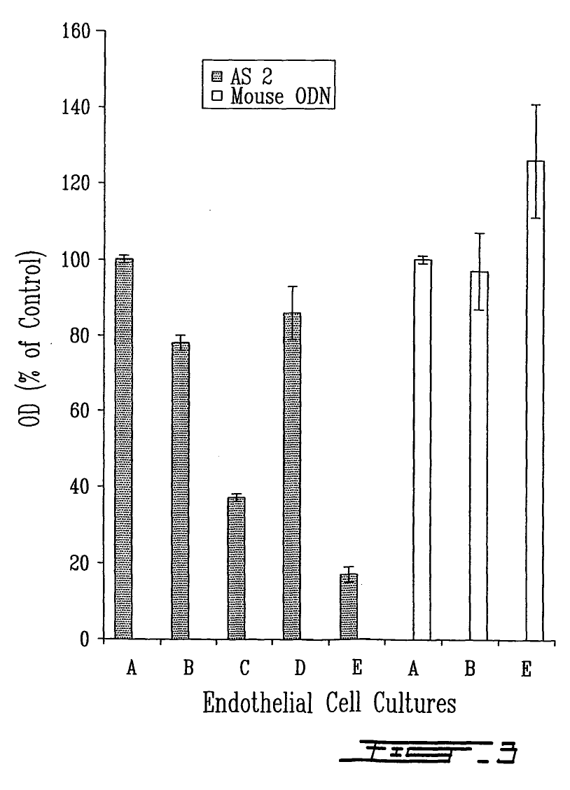

Results shown in FIG. 3 demonstrate that the effect of ODN treatment differed among different cell cultures and ranged from no reduction to an 80% reduction in the level of αᵥ expression in cells treated with AS2. A murine αᵥ antisense ODN showed no effect. In FIG. 3, HUVEC were treated with 40 μM ODN for 48 h and seeded into gelatin-coated 96-well plates at a density of 5×10³ cells/well; αᵥβ₃ expression was determined 24 h later with ELISA as described with respect to FIG. 1. Mouse αᵥ antisense ODN was used as a control; results are expressed as percent expression relative to untreated cells; and endothelial cell cultures A-E were derived from five individual cords.

### EXAMPLE III

### Determination of Viability of Cells Treated with αᵥ Antisense Oligonucleotides

MTT Assay

To determine cell viability after treatment with the oligonucleotides, the cells were seeded onto 0.3% gelatin-coated 96-well tissue culture plates (Sarstedt) at a density of 5×10³ cells per well in 200 μl medium containing 5-40 μM antisense ODN. At the intervals indicated in the text, 10 μl of a 5 mg/ml MTT (3-(4,5-dimethylthiazol-2-yl)-2,5-diphenyl tetrazolium bromide) (Sigma) solution were added to each well and the plates were incubated for 4 h at 37° C. To each well, 160 μl of dimethyl sulfoxide (DMSO) (Fisher) were added and the wells mixed thoroughly to dissolve the dark blue formazan crystals formed. Color intensity was measured with a ThermoMax™ Microplate reader (Molecular Devices Corporation) at a wavelength of 550 nm. The program used to analyze the data was SOFTmax™ 2.32 Software package for the MAXline™ Microplate Readers (Molecular Devices Corporation).

To confirm that the inhibitory effect of the ODN was not due to non-specific cytotoxic effects, cell survival was assessed after ODN treatment using the MTT assay. Following treatment with 5-40 μM ODN, the cells were plated into gelatin-coated 96-well plates and cell survival was analyzed under different culture conditions. In the presence of medium containing 1% FCS, after treatment with AS2, no significant cell death was observed relative to untreated cells under identical conditions. Although some cell lifted due to reduction in cell adhesiveness.

### EXAMPLE IV

### Inhibition of Cell Migration by αᵥ Antisense Oligonucleotides

The effect of αᵥ antisense ODN on cell migration is herein analyzed.

Endothelial Cell Migration Assay

Cell migration was measured using 8.0-μm nucleopore filters (Fisher) pre-coated with 0.3% gelatin. The filters were placed into 24-well tissue culture plates (Sarstedt) and 4×10⁴ cells in Medium-199 containing 0.1% BSA were evenly loaded onto each filter. VEGF at a concentration of 25 ng/ml with 10 μg/ml human fibronectin (Roche, Burlington, Ontario, Canada) were placed in the lower chamber to induce cell motility. Cell migration was measured 8-24 h later. Cells were fixed with 0.125% glutaraldehyde for 20 min and stained with 0.5% crystal violet (Fisher). All non-migrating cells were removed from the upper face of the filter with a cotton swab. Migrating cells on the lower surface of the filter were enumerated using a Nikon Diaphot-TMD™ inverted microscope (Nikon Canada, Montreal, Quebec, Canada) equipped with an ocular square millimeter grid. To assess the effects of a, antisense ODN on endothelial cell migration, HUVEC were treated with 5-40 μM ODN for 48 h at 37° C. prior to loading of the cells onto gelatin-coated nucleopore filters.

HUVEC Migration in Response to VEGF and bFGF

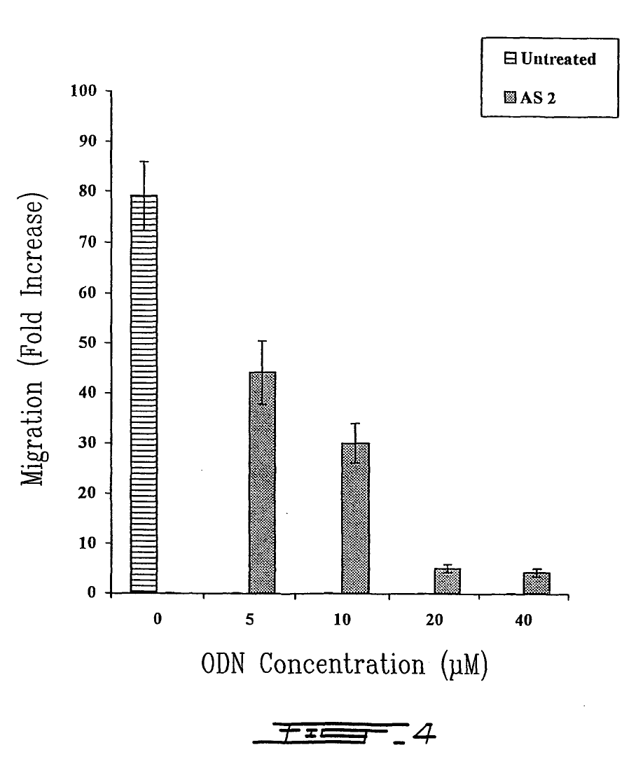

Results shown in FIG. 4 demonstrate that the inhibition of HUVEC migration was dose-dependent and that migration could be reduced by up to 95% at 40 μM, relative to untreated controls. In FIG. 4, HUVEC were treated with 5-40 μM ODN for 48 h at 37° C.; cells were dispersed and loaded onto gelatin-coated nucleopore filters for a 24 h incubation at 37° C. in the presence of VEGF and fibronectin; results are expressed as fold increase in migration relative to cells incubated in the absence of VEGF and fibronectin.

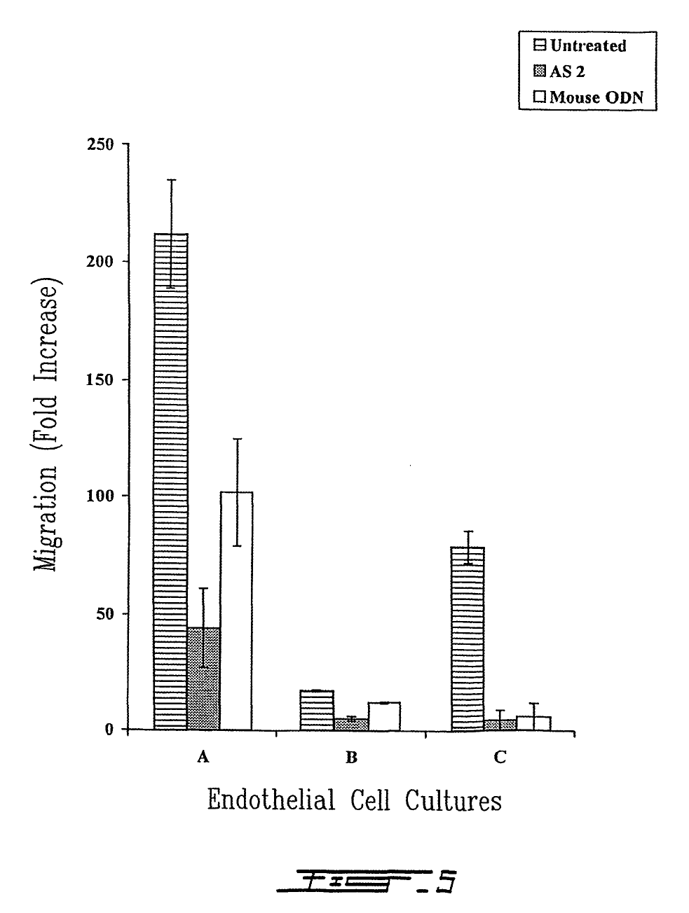

The results shown in FIG. 5 demonstrate that cells derived from different umbilical cords varied in the magnitude of their response to migration-inducing factors as well as in their sensitivity to the inhibitory effects of both human (AS2) and murine (Mouse ODN) αᵥ antisense ODN. The difference in the reduction caused by AS2 and Mouse ODN was significant for endothelial cell cultures A (p<0.0005) and B (p<0.005). In FIG. 5, HUVEC were treated with ODN and seeded onto nucleopore filters as described with respect to FIG. 4; mouse αᵥ antisense ODN was used under the same conditions; endothelial cell cultures A, B and C were isolated from three individual cords; results are expressed as fold increase in migration relative to cells not induced by migration factors and are based on 2 filters per migration assay: cells were used after three in vitro passages; the difference in the reduction caused by human and murine αᵥ antisense ODN was significant for endothelial cell cultures A (p<0.0005) and B (p<0.005).

### EXAMPLE V

### Inhibition of Thymidine Incorporation by αᵥ Antisense Oligonucleotide

The effect of αᵥ antisense ODN on DNA synthesis in response to bFGF was next examined.

Endothelial Cell Proliferation Assay

HUVEC were treated with the oligonucleotides first in complete HUVEC medium and 24 h later, in Medium-199 supplemented with 1% FCS. On the following day, the cells were dispersed with 0.05% trypsin-EDTA and seeded at a density of 5×10³ cells per well in 96-well tissue culture plates pre-coated with 0.3% gelatin. The cells were incubated for 48-72 h at 37° C. in Medium-199 containing 1% FCS and bFGF at a concentration of 20 ng/ml. The cells were pulsed for 18 h with 1.0 μCi/ml [³H]-thymidine (50-90 mCi/mmol, Mandel Scientific Company Ltd., Guelph, Ontario, Canada), lysed by repeated freezing and thawing, harvested using the Skatron Cell Harvester (Skatron Instruments Inc., Sunnyvale, Calif., USA) and absorbed onto Filtermats filter papers (Skatron Instruments Inc.). The Filtermats were added into scintillation tubes (Skatron Instruments Inc.) containing 3 ml of CytoScint scintillation cocktail (ICN, Costa Mesa, Calif., USA) and radioactivity was measured in a LKB 1217 Rackbeta liquid scintillation counter (LKB, Helsinki, Finland). Cells incubated in HUVEC medium or in Medium-199 containing 1% FCS served as controls for maximal or baseline uptake levels, respectively.

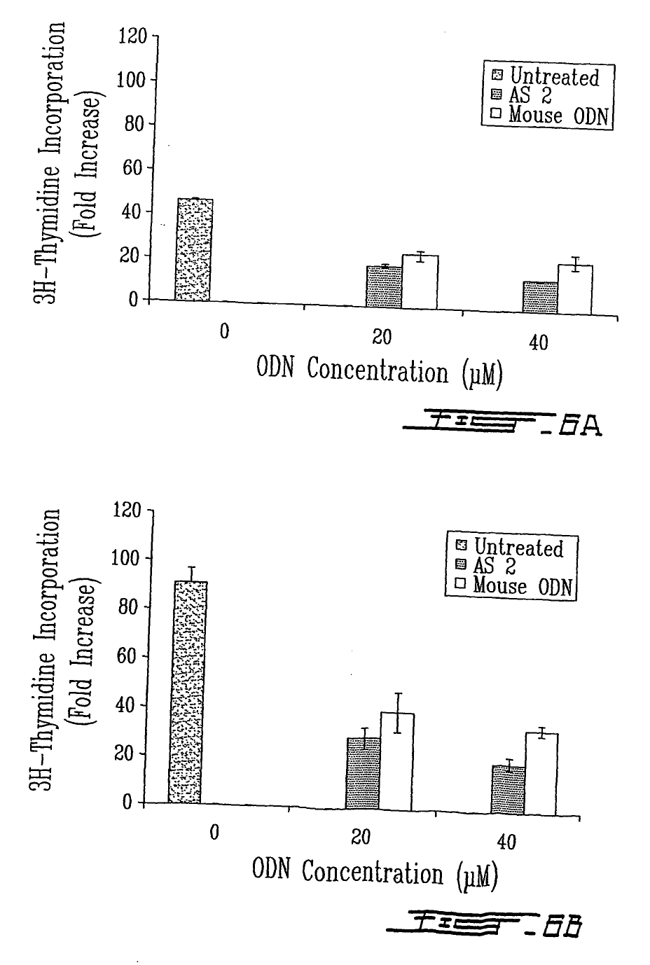

To test effect of anisense ODN on DNA synthesis HUVEC were pretreated with 20 or 40 μM ODN for a total of 48 h and transferred to gelatin-coated 96-well plates for the ³H-thymidine uptake assay performed as described above. At a concentration of 40 μM, AS2 ODN reduced cell proliferation by up to 69% and 78% following 48 and 72 h incubations respectively (See FIGS. 6A and 6B). These reductions were significantly higher (p<0.05) than those seen following treatment with control ODN). In FIGS. 6A and 6B, HUVEC were pretreated with 20 or 40 μM ODN for a total of 48 h and transferred to gelatin-coated 96-well plates. The cells were incubated for 48(A) or 72(B) h in medium containing 1% serum and 20 ng/ml bFGF. ³H-thymidine incorporation was measured after an 18 h pulse. Results are expressed as fold increase in ³H-thymidine incorporation relative to cells cultured in the absence of bFGF.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

### LIST OF REFERENCES

1. Altman K. -H. et al. Chimia 50: 168, 1996.

2. Bennett C. F. et al. J. Lip. Res. 3:85, 1993.

3. Boehm T. et al. Nature 390:404, 1997.

4. Brooks P. C. et al. Science 264:569, 1994a.

5. Brooks P. C. et al. Cell 749:1157, 1994b.

6. Brooks P. C. et al. Cell 85:683, 1996.

7. Cheresh D. A. Adv. Mol. Cell. Biol. 6:225, 1993.

8. Cotter F. E. et al. Oncogene 9:3049, 1994.

9. Dvorak H. F. et al. Ann. NY Acad. Sci. 667:101, 1992.

10. Ferrara N. et al. Endocr Rev 13:18, 1992.

11. Fitzgerald L. et al. Biochem 26:8158, 1987.

12. Folkman J., Haudenschild C. C. Nature 288:551, 1980.

13. Folkman J., Shing Y. J Biol Chem 267:10931, 1992.

14. Friedlander M. et al. Science 270:1500, 1995.

15. Friedlander M. et al. Proc. Natl. Acad. Sci. USA 93:9764, 1996.

16. Giles R. V. et al. Antisense Res. Dev. 5:23, 1995.

17. Glaser B. M. et al. J. Cell. Biol. 60:673, 1980.

18. Goldsmith J. C., et al. Lab Invest 51:643, 1984.

19. Gross J. L. et al. Proc. Natl. Acad. Sci. USA 80:2623, 1983.

20. Hijiya N. et al. Proc. Natl. Acad. Sci. USA 91:4499, 1994.

21. Kim K. J. et al. Nature 362:841, 1993.

22. Kisner D. Fourth Annual International Symposium on Oligonucleotide and Gene Therapy-Based Antisense Therapeutics with New Application for Genomics, San Diego, 1997.

23. Klagsbrun M., Baird A. Cell 67:229, 1991.

24. Kumar R. et al. Int. J. Oncology 12:749-757, 1998.

25. Lehmann M. et al. Cancer Res 54:2102, 1994.

26. Lisziewicz J. et al. Proc. Natl. Acad. Sci. USA 91:7942, 1994.

27. Luscinskas F. W., Lawler J. FASEB J 8:919, 1994.

28. Maciag T. et al. J Cell Biol 91:420, 1981.

29. Manoharan M. et al. Annals NY Acad Sci 660:306, 1992.

30. McGill S. N. et al. World J Surg 22:171, 1998.

31. Miele M. E. et al. Exp Cell Res 214:231, 1994.

32. Milligan J. F. et al. J Med Chem 36:1923, 1993.

33. Monia B. P. et al. Nat Med 2:668, 1996b.

34. Monia B. P. et al. J Biol Chem 271:14533, 1996a.

35. Monia B. P. et al. J Biol Chem 268:14514, 1993.

36. Nagy J. et al. Cancer Res 55:376, 1995.

37. Neufeld G. et al. Prog Growth Factor Res 5:89, 1994.

38. Reuning U. et al. Int. J. Oncology 13:893-906, 1998.

39. Senger D. R. et al. Am J Path 149:293, 1996.

40. Suzuki S. et al. J Bio Chem 262:14080, 1987.

41. Tischer E. et al. J Biol Chem 266:11947, 1991.

42. Vlodavsky I. et al. Cancer Metastasis Rev 3:203, 1990.

43. Wagner R W Nature, 372:333-335, 1994

44. Yacyshyn B. R. et al. Gastroenterology 114:1133, 1998.

45. Yeo T. -K., Dvorak H. F. In Diagnostic Immunopathology pp 685-697. Raven Press, New York, 1995.

What is claimed is:

1. An antisense oligonucleotide directed to αᵥ subunit of an αᵥβ₃ or αᵥβ₅ integrin vitronectin receptor, said antisense oligonucleotide blocking synthesis of said integrin vitronectin receptor on a target cell, thereby inhibiting angiogenesis and/or metastasis.

2. An antisense oligonucleotide according to claim 1, wherein said integrin vitronectin receptor consists of αᵥβ₃.

3. An antisense oligonucleotide according to claim 2, wherein said antisense oligonucleotide has a length of from 17 bases to 25 bases.

4. An antisense oligonucleotide according to claim 3, wherein said antisense oligonucleotide has a length of 18 bases and is complementary to bases 31-48 of a sequence of a human αᵥ subunit encoding a signal peptide of a αᵥ molecule as set forth in SEQ ID NO: 6.

5. An antisense oligonucleotide according to claim 4, wherein said antisense oligonucleotide has a sequence as set forth in SEQ ID NO:2.

6. An antisense oligonucleotide according to claim 1, wherein said target cell is a vascular endothelial cell.

7. An antisense oligonucleotide according to claims 1,2,3,4,5 or 6, wherein said antisense oligonucleotide is an antisense phophorothioate oligonucleotide.

8. A method for blocking angiogenesis in a patient, said method comprising delivering an effective amount of an antisense oligonucleotide as defined in claim 1, 2, 3, 4, 5, 6 or 7 to target cell of said patient, thereby blocking synthesis of integrin vitronectin receptor on said target cell and blocking angiogenesis.

9. A method according to claim 8, wherein said patient has a disease selected from the group consisting of diabetic retinopathy, rheumatoid arthritis, chronic inflammation and cancer.

10. A method for blocking metastasis from a primary site of a tumor in a patient, said method comprising delivering an efficient amount of an antisense oligonucleotide as defined in claim 1, 2, 3, 4, 5, 6 or 7 to target cell of said patient, thereby inhibiting expression of adhesion molecules by said target cell and blocking metastasis from said primary site of said tumor in said patient.

11. Use of an antisense oligonucleotide as defined in claim 1, 2, 3, 4, 5, 6 or 7 for blocking angiogenesis in a patient.

12. The use as claimed in claim 11, wherein said patient is suffering from a disease selected from the group consisting of diabetic retinoplathy, rheumatoid arthritis, chronic inflammation and cancer.

13. Use of an antisense oligonucleotide as defined in claim 1 or blocking metastasis from a primary site of a tumor in a patient.

14. Use of an antisense as defined in claim 1, 2, 3, 4, 5, 6 or 7 for manufacturing a medicament for blocking angiogenesis in a patient.

15. The use as claimed in claim 14, wherein said patient is suffering from a disease selected from the group consisting of diabetic retinoplathy, rheumatoid arthritis, chronic inflammation and cancer.

16. Use of an antisense as defined in claim 1, 2, 3, 4, 5, 6 or 7 for manufacturing a medicament for blocking metastasis from a primary site of a tumor in a patient.


SEQUENCE LISTING (6 sequences)

SEQ ID NO: 1 - DNA, 18 bp, Artificial Sequence
  antisense complementary to the human alpha-v subunit sequence
tcagcatcaatatcttgt

SEQ ID NO: 2 - DNA, 18 bp, Artificial Sequence
  antisense complementary to the human alpha-v subunit sequence
aagccatcgccgaagtgc

SEQ ID NO: 3 - DNA, 18 bp, Artificial Sequence
  antisense complementary to the human alpha-v subunit sequence
gactgtccacgtctaggt

SEQ ID NO: 4 - DNA, 18 bp, Artificial Sequence
  antisense complementary to the human alpha-v subunit sequence
gcacttcggcgatggctt

SEQ ID NO: 5 - DNA, 20 bp, Artificial Sequence
  antisense complementary to the human alpha-v subunit sequence
caaggtcgcacaccacctgc

SEQ ID NO: 6 - DNA, 131 bp, Artificial Sequence
  sequence from vitronectin receptor alpha subunit
ggctaccgctcccggcttggcgtcccgcgcgcacttcggcgatggcttttccgccgcggcgacggctgcgcctcggtccccgcggcctcccgcttcttctctcgggactcctgctacctctgtgccgcgcc